(19) United States
(12) Patent Application Publication
(10) Pub. No.: US 2011/0144674 A1
(43) Pub. Date: Jun. 16, 2011

(54) DEBRIDEMENT CUTTING HEADS, METHODS, AND SYSTEMS EMPLOYING REDUCED PRESSURE

(76) Inventors: Keith Patrick Heaton, Poole (GB); James Joseph Sealy, HIGH WYCOMBE (GB)

(21) Appl. No.: 12/903,664

(22) Filed: Oct. 13, 2010

Related U.S. Application Data
(60) Provisional application No. 61/252,371, filed on Oct. 16, 2009.

(51) Int. Cl. A61B 17/32

(52) U.S. Cl. 606/167

(57) ABSTRACT

Apparatuses, systems, and methods involve using reduced pressure to position a portion of tissue in a cutting head and cutting the tissue to debride a tissue site. A cutting head includes a debridement conduit having a distal end and a proximal end. A portion of the debridement conduit adjacent to the distal end comprises a tissue treatment area. The cutting head also includes an opening to the tissue treatment area formed at the distal end of the debridement conduit. The tissue treatment area is adapted to receive the portion of tissue through the opening under reduced pressure. The cutting head also includes a selectively moveable cutting member associated with the debridement conduit at the tissue treatment area. The cutting member is for cutting the portion of tissue from the tissue site while the portion of tissue resides in the debridement conduit. Other methods, systems, and apparatuses are disclosed.

### RELATED APPLICATION

The present invention claims the benefit, under 35 USC §119(e), of the filing of U.S. Provisional Patent Application Ser. No. 61/252,371 entitled “Debridement Cutting Heads, Methods, and Systems Employing Reduced Pressure,” filed Oct. 16, 2009, which is incorporated herein by reference for all purposes.

### BACKGROUND

The present invention relates generally to medical treatment systems, and more particularly, to tissue debridement systems and methods.

Necrotic tissue may retard wound healing. As such, it may at times be desirable to remove necrotic tissue. The therapeutic intervention for necrotic tissue presenting in the wound is debridement. A number of general approaches exist for debridement, e.g., mechanical, enzymatic or chemical, sharp, biosurgical, and autolytic.

### SUMMARY

According to an illustrative, non-limiting embodiment, a method for cutting a portion of tissue from a tissue site of a patient using reduced pressure includes providing a debridement conduit having a distal end and a proximal end, applying a reduced pressure to the portion of tissue via the debridement conduit, and causing the portion of tissue to enter the distal end of the debridement conduit in response to applying the reduced pressure. The method also includes cutting the portion of tissue in the debridement conduit from the tissue site.

According to another illustrative, non-limiting embodiment, a system for cutting a portion of tissue from a tissue site of a patient using reduced pressure includes a reduced-pressure source for supplying reduced pressure. The system includes a cutting head for placement at the tissue site that is selectively in fluid communication with the reduced-pressure source. The cutting head includes a debridement conduit having a distal end and a proximal end. A portion of the debridement conduit adjacent to the distal end comprises a tissue treatment area. The cutting head also includes an opening to the tissue treatment area formed at the distal end of the debridement conduit. The tissue treatment area of the debridement conduit is adapted to receive the portion of tissue through the opening under reduced pressure. The cutting head also includes a cutting member associated with the debridement conduit at the tissue treatment area. The cutting member is movable and has a receiving position and a cutting position to cut the portion of tissue from the tissue site while the portion of tissue resides in the tissue treatment area of the debridement conduit. The system includes a canister fluidly coupled to the cutting head and the reduced-pressure source. The reduced pressure draws cut tissue from the debridement conduit into the canister.

According to another illustrative, non-limiting embodiment, an apparatus for cutting a portion of tissue from a tissue site of a patient using reduced pressure includes a cutting head for placement at the tissue site. The cutting head includes a debridement conduit having a distal end and a proximal end. A portion of the debridement conduit adjacent to the distal end comprises a tissue treatment area. The cutting head also includes an opening to the tissue treatment area formed at the distal end of the debridement conduit. The tissue treatment area of the debridement conduit is adapted to receive the portion of tissue through the opening under reduced pressure. The cutting head also includes a cutting member associated with the debridement conduit at the tissue treatment area. The cutting member is movable and has a receiving position and a cutting position to cut the portion of tissue from the tissue site while the portion of tissue resides in the tissue treatment area of the debridement conduit.

According to another illustrative, non-limiting embodiment, a method for cutting a portion of tissue from a tissue site of a patient using reduced pressure includes applying a cutting head to the tissue site. The cutting head includes a debridement conduit having a distal end and a proximal end. A portion of the debridement conduit adjacent to the distal end comprises a tissue treatment area. The cutting head also includes an opening to the tissue treatment area formed at the distal end of the debridement conduit. The tissue treatment area of the debridement conduit is adapted to receive the portion of tissue through the opening under reduced pressure. The cutting head also includes a cutting member associated with the debridement conduit at the tissue treatment area. The cutting member is movable and has a receiving position and a cutting position to cut the portion of tissue from the tissue site while the portion of tissue resides in the tissue treatment area of the debridement conduit. The method also includes supplying reduced pressure to the cutting head to cause the portion of tissue to enter the tissue treatment area of the debridement conduit, and cutting the portion of tissue from the tissue site using the cutting member.

According to another illustrative, non-limiting embodiment, a method of manufacturing an apparatus for cutting a portion of tissue from a tissue site of a patient using reduced pressure includes forming a cutting head for placement at the tissue site. The cutting head includes a debridement conduit having a distal end and a proximal end. A portion of the debridement conduit adjacent to the distal end comprises a tissue treatment area. The cutting head also includes an opening to the tissue treatment area formed at the distal end of the debridement conduit. The tissue treatment area of the debridement conduit is adapted to receive the portion of tissue through the opening under reduced pressure. The cutting head also includes a cutting member associated with the debridement conduit at the tissue treatment area. The cutting member is movable and has a receiving position and a cutting position to cut the portion of tissue from the tissue site while the portion of tissue resides in the tissue treatment area of the debridement conduit.

### BRIEF DESCRIPTION OF THE DRAWINGS

FIG. 1 is a schematic, side view, with a portion shown in cross-section and another portion shown as a diagram, of a debridement system according to one illustrative, non-limiting embodiment;

FIG. 2 is a schematic, cross-sectional view of the cutting head of FIG. 1 with a cutting member in a cutting position and a portion of tissue severed from the tissue site;

FIG. 3 is a schematic, perspective view of the cutting head of FIG. 1;

FIG. 4 is a schematic, side view, with a portion shown in cross-section, of a handle and a cutting head, including an elastic cover, according to one illustrative, non-limiting embodiment;

FIG. 5 is a schematic, side view of the cutting head of FIG. 4 shown with the elastic cover applied to the tissue treatment area of the debridement conduit of the cutting head; and

FIG. 6 is a schematic, end view of the cutting head of FIG. 5.

### DETAILED DESCRIPTION

In the following detailed description of the illustrative embodiments, reference is made to the accompanying drawings that form a part hereof. These embodiments are described in sufficient detail to enable those skilled in the art to practice the invention, and it is understood that other embodiments may be utilized and that logical structural, mechanical, electrical, and chemical changes may be made without departing from the spirit or scope of the invention. To avoid detail not necessary to enable those skilled in the art to practice the embodiments described herein, the description may omit certain information known to those skilled in the art. The following detailed description is, therefore, not to be taken in a limiting sense, and the scope of the illustrative, non-limiting embodiments are defined only by the appended claims.

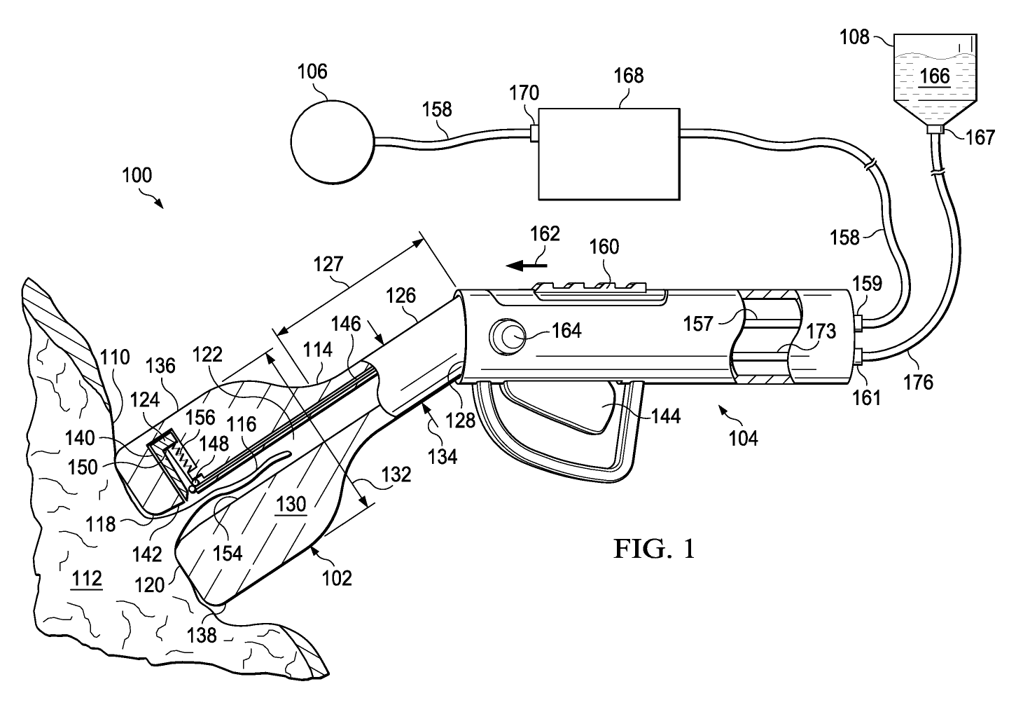

Referring now primarily to FIGS. 1 through 3, an illustrative, non-limiting embodiment of a debridement system 100, or other tissue removal system 100, includes a cutting head 102 and handle 104 that are fluidly coupled to a reduced-pressure source 106 and a fluid source 108. The handle 104, which is coupled to the cutting head 102, is used to apply the cutting head 102 to a wound 110 on a tissue site 112 so that a portion of tissue, e.g., portion 116 of tissue, can be drawn into an opening 118 of a debridement conduit 114 using reduced pressure and cut from the tissue site 112. The debridement system 100 uses reduced pressure and the cutting head 102 to debride the tissue site 112. The tissue site 112 may be the bodily tissue of any human, animal, or other organism, including bone tissue, adipose tissue, muscle tissue, dermal tissue, vascular tissue, connective tissue, cartilage, tendons, ligaments, or any other tissue. The wound 110 on the tissue site 112 may be due to a variety of causes, including trauma, surgery, disease, etc.

When the cutting head 102 is applied to the tissue site 112, the debridement conduit 114, which forms part of the cutting head 102, receives the portion 116 of tissue through the opening 118 that is at a distal end 120 of the debridement conduit 114. The portion of the debridement conduit 114 in which the portion 116 of tissue generally resides upon entry through the opening 118 is a tissue treatment area 122. The reduced pressure from the reduced-pressure source 106 may be communicated to the tissue site 112 through the debridement conduit 114 to draw the portion 116 of tissue into the tissue treatment area 122. Examples of treatments that can occur in the tissue treatment area 122 include removal of the portion 116 of tissue (debridement) or fluids (e.g., ascites, exudate, blood, etc.), or application of reduced pressure to the tissue site 112. As used herein, unless otherwise indicated, “or” does not require mutual exclusivity. Once the portion 116 of tissue resides in the tissue treatment area 122 of the debridement conduit 114, a cutting member 124, or cutting mechanism, may be moved, or activated, from a receiving position to a closed position or cutting position to cut, or sever, the portion 116 of tissue from the tissue site 112. After activation, the cutting member 124 may return to a receiving position.

The term “reduced pressure” as used herein generally refers to a pressure less than the ambient pressure at a tissue site that is being subjected to treatment. In most cases, this reduced pressure will be less than the atmospheric pressure at which the patient is located. Alternatively, the reduced pressure may be less than a hydrostatic pressure associated with tissue at the tissue site 112. Although the terms “vacuum” and “negative pressure” may be used to describe the pressure applied to the tissue site 112, the actual pressure reduction applied to the tissue site 112 may be significantly less than the pressure reduction normally associated with a complete vacuum. Unless otherwise indicated, values of pressure stated herein are gauge pressures. Similarly, references to increases in reduced pressure typically refer to a decrease in absolute pressure, while decreases in reduced pressure typically refer to an increase in absolute pressure.

The debridement conduit 114 may include both the tissue treatment area 122 and an extender portion 126. The tissue treatment area 122 is adjacent to or helps form the distal end 120 of the debridement conduit 114, and the extender portion 126 is adjacent to or helps form a proximal end 128 of the debridement conduit 114. The extender portion 126 may be any length, and may be used to provide a distance 127 between the tissue treatment area 122 and the handle 104. The distance 127 may be varied for a variety of purposes, such as extending the reach of the cutting head 102.

The tissue treatment area 122 of the debridement conduit 114 may include an enlarged cushion portion 130. The enlarged cushion portion 130 may taper in width (diameter) toward the extender portion 126 of the debridement conduit 114. The enlarged cushion portion 130 may serve any of a variety of purposes. For example, the enlarged cushion portion 130 may have elastic, or cushion-like, properties so that the distal end 120 of the cutting head 102 does not cause abrasion or injury to the wound 110, or the tissue site 112, to which the cutting head 102 is applied. Thus, the enlarged cushion portion 130 allows the cutting head 102 to come into full contact with the wound 110 or the tissue site 112, if necessary, when severing the portion 116 of tissue from the tissue site 112. The enlarged cushion portion 130 may also prevent or reduce friction between the distal end 120 of the cutting head 102 and the wound 110 or the tissue site 112.

The enlarged cushion portion 130 has an average width 132 (or outside diameter) that is greater than an average width 134 (or outside diameter) of the extender portion 126. The width 132 (W₁) of the enlarged cushion portion 130 may be larger than the width 134 (W₂) of the extender portion 126 (W₁>W₂). Moreover, the width 132 (W₁) may be greater than the width 134 (W₂) by any factor, e.g., W₁>1.5W₂, W₁>2W₂, W₁>3W₂, etc. In yet another illustrative, non-limiting embodiment, the tissue treatment area 122 of the debridement conduit 114 does not include any enlarged cushion portion 130 at all or is indistinguishable, i.e., W₁=W₂.

The enlarged cushion portion 130 may be formed from one or more elastic materials. The enlarged cushion portion 130 may, for example, be an impermeable or semi-permeable, elastomeric material. “Elastomeric” means having the properties of an elastomer. It generally refers to a polymeric material that has rubber-like properties. More specifically, most elastomers have ultimate elongations greater than 100% and a significant amount of resilience. The resilience of a material refers to the material's ability to recover from an elastic deformation. Examples of elastomers from which the enlarged cushion portion 130 may be formed include, but are not limited to, natural rubbers, polyisoprene, styrene butadiene rubber, chloroprene rubber, polybutadiene, nitrile rubber, butyl rubber, ethylene propylene rubber, ethylene propylene diene monomer, chlorosulfonated polyethylene, polysulfide rubber, polyurethane, co-polyester, and silicones.

The enlarged cushion portion 130 may help to prevent splashing from the tissue site 112 or the vicinity of the tissue treatment area 122. For example, when the portion 116 of tissue is cut from the tissue site 112, the enlarged cushion portion 130 may prevent blood from splashing away from the tissue site 112. In another illustrative, non-limiting embodiment, the enlarged cushion portion 130 may also create a fluid seal when applied to the tissue site 112 to help target or strengthen the reduced pressure that is applied to the tissue site 112 or vicinity. “Fluid seal” means a seal adequate to maintain reduced pressure at a desired site given the particular reduced-pressure subsystem involved. In one embodiment, the enlarged cushion portion 130 is transparent to visually expose the portion 116 of tissue in the tissue treatment area 122.

The opening 118 at the distal end 120 of the cutting head 102 provides access to the tissue site 112. Forming the opening 118 at the distal end 120, as opposed to sides 136 and 138 of the cutting head 102, provides a direct route through which to apply reduced pressure to the tissue site 112, and therefore aids in selecting the portion 116 of tissue that enters the tissue treatment area 122 by adjusting the reduced pressure transferred to the tissue site 112. The diameter of the opening 118 may also be varied to control the amount of tissue entering the opening 118. In other embodiments the opening 118 may be located at the sides 136 and 138 of the cutting head 102. The opening 118 is also shown to be a tapered opening. Such tapering may help to prevent injury at the tissue site 112 by eliminating sharp edges or may help to block splashing when the portion 116 of tissue is cut from the tissue site 112. In one illustrative, non-limiting embodiment, the opening 118 may be adjustable to control an amount of tissue entering the tissue treatment area 122. A relatively larger diameter opening 118 will allow more tissue to enter and a relatively smaller diameter opening 118 will allow less tissue to enter. In one illustrative, non-limiting embodiment, the opening 118 may be controllable in real time with a variable aperture for the opening 118 and an actuator (not explicitly shown) for modifying the size of the opening 118. In this latter embodiment, the variable aperture opening 118 is an aperture that is variable between a fully opened position and a restricted position.

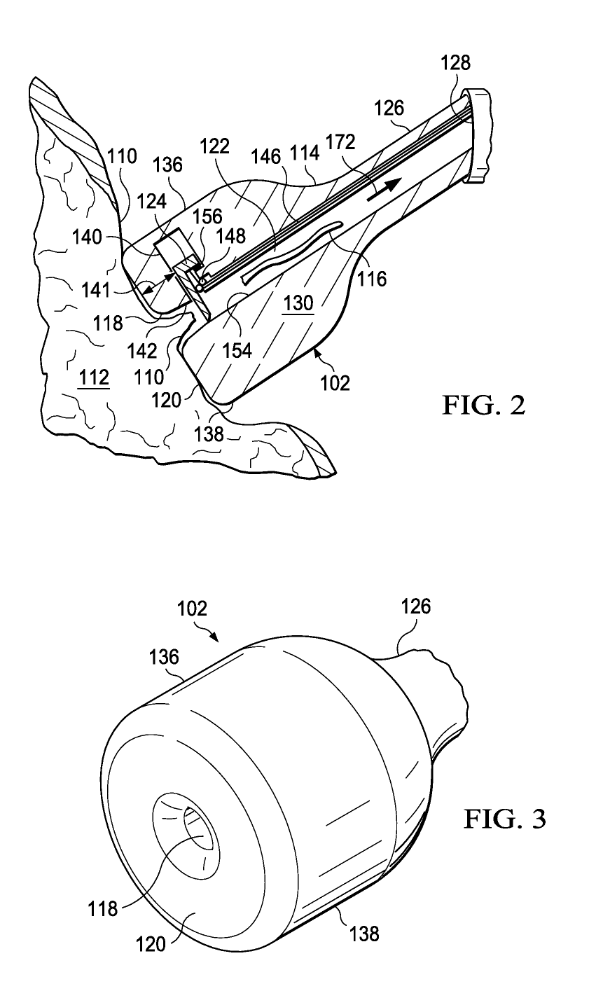

The cutting member 124 is movable between a receiving position, which is shown in one illustrative embodiment in FIG. 1, and a closed position or cutting position, which is shown in one illustrative embodiment in FIG. 2. While in the receiving position, the cutting member 124 may be substantially disposed in a recess 140 on an inner surface 142 of the tissue treatment area 122 of the debridement conduit 114 or otherwise in a position to provide adequate clearance in the tissue treatment area 122 of the debridement conduit 114 to allow the portion 116 of tissue to enter the treatment area 122. When activated to move from the receiving position to the cutting position, the cutting member 124 cuts, or severs, the portion 116 of tissue from the tissue site 112.

The cutting member 124 may take numerous forms, but is shown in the illustrative, non-limiting embodiment to be a single blade. The cutting member 124 may take several forms, however, such as two blades that close in a shutter-like manner, or any number of circular blades that swivel into the tissue treatment area 122 when in the cutting position. In one specific example, the cutting member 124 may be four circular blades that swivel into the tissue treatment area 122 when moved into the cutting position.

The cutting member 124 may be moved from the receiving position to the close position using a cutting switch 144 that is located on the handle 104. One illustrative, non-limiting example of the mechanical relation between the cutting switch 144 and the cutting member 124 is illustrated in FIGS. 1 and 2. In this non-limiting, illustrative example, pressing the cutting switch 144 pulls a wire 146 toward the handle 104. The wire 146, which passes through pulleys 148, is coupled to the cutting member 124 at a distal end 150 of the wire 146. When the wire 146 is pulled towards the handle 104, the cutting member 124 is pulled toward the tissue treatment area 122 defined by the debridement conduit 114, and thereby moves into the cutting position shown in FIG. 2.

In moving to the cutting position, the cutting member 124 moves closer to, and may come into contact with, an opposing side 154 of the inner surface 142 of the debridement conduit 114. When the cutting switch 144 is released, or moved into a non-activation position, the cutting member 124 is biased back into the recess 140 by a spring 156. In another illustrative, non-limiting embodiment, the cutting switch 144 may be electrically coupled to the cutting member 124, and use electrical signals to control the movement of the cutting member 124 in cooperation with a solenoid or other device.

The cutting switch 144 on the handle 104 is only one of several possible switches that may be located on the handle 104. The handle 104 provides a way for a user, such as a caretaker, to grip or control the cutting head 102 when performing treatment on the tissue site 112. The handle 104 includes a reduced-pressure port 159 and a fluid port 161. A reduced-pressure conduit 158 fluidly couples the reduced-pressure port 159 and the reduced-pressure source 106. A fluid conduit 176 fluidly couples the fluid source 108 and the fluid port 161. The handle 104 also includes intermediate conduits 157 and 173 within the handle that provide selective fluid communication between the cutting head 102 and either or both of a reduced-pressure port 159 and the fluid port 161, respectively.

The handle 104 may also include a reduced-pressure switch 160. The reduced-pressure switch 160 controls an amount of reduced pressure that is applied to the tissue treatment area 122 and the tissue site 112 from the reduced-pressure source 106. As the reduced-pressure switch 160 slides in the direction indicated by arrow 162, the amount of reduced pressure applied to the tissue treatment area 122 of the cutting head 102 and the tissue site 112 is increased. In one example, a valve (not explicitly shown) in the intermediate conduit of the handle 104 is controlled by the reduced-pressure switch 160, and the valve variably opens and closes in response to the position of the reduced-pressure switch 160 to determine an amount of reduced pressure that is transferred to the cutting head 102. In another example, the reduced-pressure switch 160 may be mechanically or electrically coupled to a controller (not shown) that controls the amount of reduced pressure supplied by the reduced-pressure source 106.

The handle 104 also includes a fluid switch 164 that is used to control the supply of a fluid 166 to the cutting head 102. The fluid 166 may include substances, or itself be a substance, that aids the treatment of the tissue site 112. For example, the fluid 166 may include healing factors, anesthetics, growth factors, antibiotics, etc. The fluid 166 may also be used to irrigate the tissue site 112 before, during, or after debridement of the portion 116 of tissue.

When the fluid switch 164 is pressed, or activated, the fluid 166 is allowed to flow through the debridement conduit 114 including the tissue treatment area 122, where the fluid 166 is able to contact the tissue site 112, including the portion 116 of tissue. One example of a technique by which the fluid flow may be controlled by the fluid switch 164 is by the use of a valve (not explicitly shown) in the intermediate conduit of the handle 104 that opens when the fluid switch 164 is pressed. In another example, pressing the fluid switch 164 causes a valve 167 to open at the fluid source 108, thereby allowing the fluid 166 to flow through the fluid conduit 176, the intermediate conduit 173 in the handle 104, and into the debridement conduit 114.

Any type of switch may be used for the cutting switch 144, the reduced-pressure switch 160, and the fluid switch 164, such as a toggle switch, a trigger, a button, a slidable switch, or an electronic switch. In an alternative illustrative, non-limiting embodiment, all or a portion of the switches on the handle 104 may instead be located on a separate controller (not shown) that has a user interface. In this alternative embodiment, the application of reduced pressure, the fluid 166, and the cutting member 124 is controlled through a user interface located on the controller. The controller may be included in a housing (not shown) that contains the reduced-pressure source 106 or the fluid source 108.

The debridement system 100 also includes a canister 168 on reduced-pressure conduit 158. The portion 116 of tissue removed from the tissue site 112, when severed from the tissue site 112, is delivered to the canister 168. In particular, reduced pressure from reduced-pressure source 106 draws the portion 116 of tissue that is severed from the tissue site 112 away from the tissue treatment area 122, through the intermediate conduit 157 of the handle 104 and at least a portion of the reduced-pressure conduit 158, and into the canister 168. The portion 116 of tissue, once deposited in the canister 168, may then be disposed of or used in further medical testing. The portion 116 of tissue, or any other liquid or solid substances that are deposited in the canister 168, are substantially prevented from being drawn into the reduced-pressure source 106 by a hydrophobic filter 170 that is interposed between the canister 168 and the reduced-pressure source 106.

In one illustrative, non-limiting embodiment, the reduced-pressure conduit 158 and the fluid conduit 176 may be integrated into a single delivery conduit that has two or more lumens. Of these lumens, one or more may supply reduced pressure from the reduced-pressure source 106 to the handle 104, and another one or more lumens may supply fluid 166 from the fluid source 108 to the handle 104.

In use, the debridement system 100 utilizes the cutting head 102 that is applied to the tissue site 112 using the handle 104 to cut a portion of undesired tissue, e.g., the portion 116 of tissue, from the tissue site 112. The cutting head 102 may be applied to the tissue site 112 by positioning the tissue treatment area 122 adjacent the portion of undesired tissue, e.g., the portion 116 of tissue. For example, the distal end 120 of the debridement conduit 114 may be placed against, or abut, the tissue site 112. Upon positioning the tissue treatment area 122 adjacent the tissue site 112, using the reduced-pressure switch 160, reduced pressure from the reduced-pressure source 106 may then be applied to the tissue site 112 to draw the portion of undesired tissue, e.g., the portion 116 of tissue, through the opening 118 and into the tissue treatment area 122 of the debridement conduit 114.

The reduced-pressure switch 160 may be variably moved to change the amount of reduced pressure being applied to the tissue site 112. By changing the amount of reduced pressure being applied to the tissue site 112, the portion 116 of tissue that is being drawn through the opening 118 may be increased or decreased as desired. For example, a user that desires to increase the amount of tissue that is cut from the tissue site 112 may increase the amount of reduced pressure being applied to the tissue site 112 to increase the portion size of tissue 116 that is drawn through the opening 118. This increased portion 116 of tissue may then be cut off from the tissue site 112 by activating the cutting switch 144 to activate the cutting member 124. Conversely, a user that desires to decrease the portion 116 of tissue being cut from the tissue site 112 may reduce the amount of reduced pressure being applied to the tissue site 112, and then cut this reduced amount of tissue from the tissue site 112 by activating the cutting switch 144.

In general, there is a direct correlation between the amount of reduced pressure applied and the size of the tissue entering the tissue treatment area 122. The amount of reduced pressure, and hence the amount of tissue, being drawn into the tissue treatment area 122 may be customized based on a variety of factors, such as the hardness of the tissue site 112, the amount of dead, or necrotic, tissue at the tissue site 112, or the medical necessity of cutting a particular amount of tissue. Before, during, or after cutting of the portion 116 of tissue from the tissue site 112, the fluid 166 may be supplied to the tissue site 112 by activating the fluid switch 164. Again, the amount of fluid 166 that is supplied to the tissue site 112 is controllable by the fluid switch 164.

Referring now primarily to FIG. 2, upon cutting the portion 116 of tissue from the tissue site 112, the portion 116 of tissue is drawn through the debridement conduit 114 in a direction indicated by the arrow 172. The reduced pressure draws the portion 116 of tissue from the tissue treatment area 122 into the reduced-pressure conduit 158 and into the canister 168, where the portion 116 of tissue may be disposed of or used in medical testing.

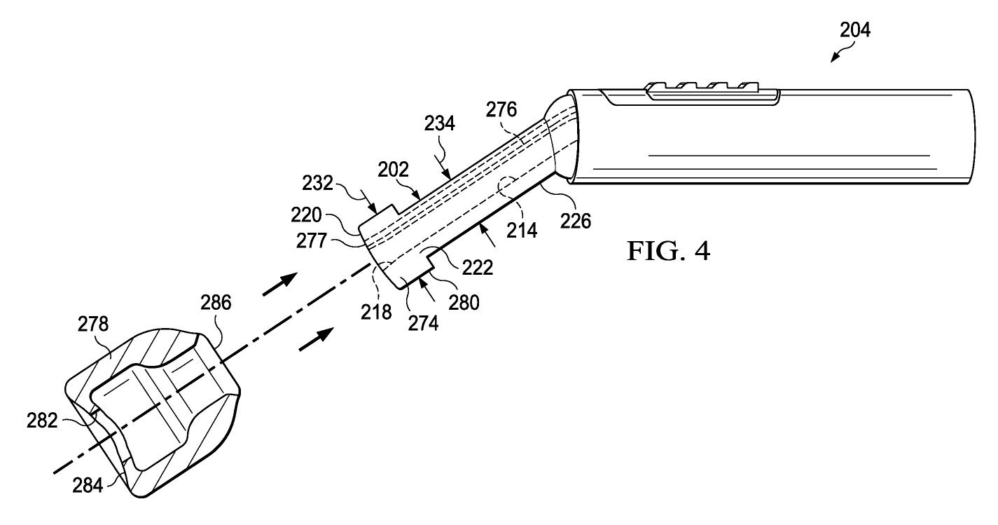

Referring now primarily to FIGS. 4 through 6, another illustrative, non-limiting embodiment of a cutting head 202 is presented. The cutting head 202 may be used as part of a debridement system, such as debridement system 100 of FIGS. 1-3. The cutting head 202 is coupled to a handle 204.

The cutting head 202 includes a tissue treatment area 222 of a debridement conduit 214 that includes an enlarged portion or enlarged disc 274 formed on a distal end 220 of the cutting head 202. In one illustrative, non-limiting embodiment, both an extender portion 226 and the enlarged disc 274 are formed from a substantially rigid material. In addition, a width 232 of the enlarged disc 274 is greater than a width 234 of the extender portion 226. The width 232 (W₃) of the enlarged disc 274 may be greater than the width 234 of the extender portion 226 (W₄), i.e., W₃>W₄, by any factor, such as W₃>1.5W₄, W₃>2W₄, W₃>3W₄, etc.

The cutting head 202 also includes a fluid supply conduit 276 that is separate from the debridement conduit 214. The fluid supply conduit 276 supplies fluid from a fluid source, such as the fluid source 108 in FIG. 1, to the distal end 220 of the cutting head 202. The fluid that is supplied by the fluid supply conduit 276 is released from the distal end 220 of the cutting head 202 via a fluid supply outlet, or port 277. As such, in contrast to the embodiment shown in FIGS. 1 through 3, the cutting head 202 may supply the fluid to a tissue site via a conduit (i.e., the fluid supply conduit 276) that is separate from the debridement conduit 214.

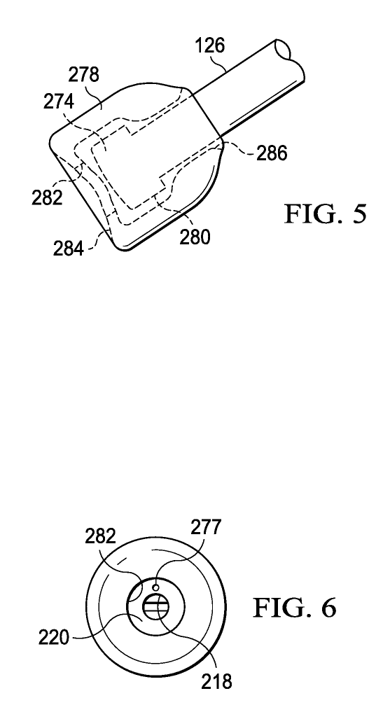

The cutting head 202 also includes an elastic cover 278 that is mateable with the enlarged disc 274. The elastic cover 278 may be placed over a distal portion 280, including the enlarged disc 274, of the cutting head 202. The elastic cover 278 may be stretched to go over the enlarged disc 274. The elastic cover 278 includes an aperture 282 at a distal end 284 of the elastic cover 278. When the distal portion 280 of the cutting head 202 is placed into a proximal opening 286 of the elastic cover 278 such that the elastic cover 278 covers the distal portion 280 as shown in FIG. 5, an opening 218 and the fluid supply port 277 are exposed through the aperture 282 (see FIG. 6). The elastic cover 278 performs a similar function to the enlarged cushion portion 130 in FIG. 1, such as performing a cushioning and splash-blocking function. Because the elastic cover 278 is removable, the elastic cover 278 may be disposed of, replaced, or substituted with a different type of elastic cover.

As an alternative or in addition, the elastic cover 278 may be chilled and placed on the cutting head 202 at the time of use or otherwise chilled. The chilled surface of the elastic cover 278 may be used to provide an analgesic effect to enhance patient comfort. The elastic cover 278 may be chilled by forming the elastic cover 278 or a portion thereof from a material, such as a gel, that may be cooled in a refrigerating unit and cooled well below the temperature of the patient. The elastic cover 278 may be chilled by forming the elastic cover 278 with cooling conduits (not shown) within the elastic cover 278 and fluidly coupling a coolant-delivery conduit that delivers a cooling medium to the cooling conduits of the elastic cover 278 to lower the temperature of the elastic cover 278.

As used herein, the term “coupled” includes coupling via a separate object and includes direct coupling. The term “coupled” also encompasses two or more components that are continuous with one another by virtue of each of the components being formed from the same piece of material. Also, the term “coupled” may include chemical, such as via a chemical bond, mechanical, thermal, or electrical coupling. “Fluid coupling” means that fluid is in communication between the designated parts or locations.

While the illustrative, non-limiting embodiments herein present the application of systems, apparatuses, and methods in the context of debridement, it should be understood that the systems, apparatuses, and methods may be used in other applications, such as obtaining tissue for a biopsy. With reference primarily to FIG. 1, the cutting head 102 of the debridement system 100 may be used to cut a portion of tissue that is delivered to the canister 168. The tissue may be removed from the canister 168 for testing.

The debridement system 100 allows regulated pressure from the reduced-pressure source 106 along with the dimensions of the cutting head 102 to limit the depth or volume of tissue 116 that can be removed by the cutting member 124. For example, a distance 141 (FIG. 2) of the cutting member 124 from the distal end 120 influences the amount of tissue removed. In some illustrative, non-limiting embodiments the distance 141 may be about 0.5 mm, 1 mm, 2 mm, 3 mm, 4, mm, 5 mm, 6 mm, 7, mm, 8 mm, 9 mm, 10 mm, 15 mm, 20 mm, 30 mm, or any number therebetween. It will be appreciated that the reduced pressure from the reduced-pressure source 106 is used in the cutting operation itself and not merely as a debris removal. The reduced pressure provided may also provide a means of removing slough and bio-film from the tissue site 112, e.g., wound, without the need for further instruments or equipment. Moreover, the debridement system 100 may be used quickly to remove non-viable or undesired tissue. The debridement system 100 may be disposable.

While the debridement system 100 is shown with a reduced-pressure source 106 that appears as a standalone unit, it should be understood that numerous sources of reduced pressure may be used. The reduced-pressure source 106 may be any device for supplying a reduced pressure, such as a vacuum pump, wall suction, micro-pump, or other source.

The present invention and its advantages have been disclosed in the context of certain illustrative, non-limiting embodiments. The illustrative descriptions above are not intended to be exhaustive or to limit the invention to the precise forms disclosed. Moreover, it should be understood that various changes, substitutions, permutations, and alterations can be made without departing from the scope of the invention as defined by the appended claims. It will be appreciated that any feature that is described in a connection to any one embodiment may also be applicable to any other embodiment.

1. A system for cutting a portion of tissue from a tissue site of a patient using reduced pressure, the system comprising:
a reduced-pressure source for supplying reduced pressure;
a cutting head for placement at the tissue site and selectively in fluid communication with the reduced-pressure source, wherein the cutting head comprises:
a debridement conduit having a distal end and a proximal end, wherein a portion of the debridement conduit adjacent to the distal end comprises a tissue treatment area,
an opening to the tissue treatment area at the distal end of the debridement conduit, wherein the tissue treatment area of the debridement conduit is adapted to receive the portion of tissue through the opening under reduced pressure, and
a cutting member associated with the debridement conduit at the tissue treatment area, the cutting member movable from a receiving position to a cutting position to cut the portion of tissue from the tissue site while the portion of tissue resides in the tissue treatment area of the debridement conduit, and wherein the cutting member is displaced from the distal end of the debridement conduit to control an amount of tissue that will be cut;
a canister fluidly coupled to the cutting head and the reduced-pressure source, wherein reduced pressure draws cut tissue from the debridement conduit into the canister; and
a delivery conduit providing fluid communication between the canister and the cutting head.

2. The system of claim 1, further comprising:
wherein the debridement conduit further comprises an extender portion adjacent the proximal end of the cutting head; and
a handle coupled to the extender portion of the debridement conduit, the handle comprising an intermediate conduit providing fluid communication between the delivery conduit and the cutting head.

3. The system of claim 1, wherein the debridement conduit comprises an extender portion having a width (W₂) adjacent the proximal end of the cutting head, wherein the tissue treatment area of the debridement conduit comprises an enlarged cushion portion having a width (W₁), wherein W₁>W₂.

4. (canceled)

5. The system of claim 1, wherein:
the tissue treatment area of the debridement conduit comprises an enlarged disc having a width (W₁);
the debridement conduit comprises an extender portion, having a width (W₂), adjacent the proximal end of the cutting head;
W₁>W₂; and
the cutting head further comprises an elastic cover adapted to cover at least a portion of the enlarged disc, the elastic cover having an aperture at a first end for exposing the opening when the elastic cover is placed on the enlarged disc.

6. (canceled)

7. The system of claim 1, wherein the cutting member is a blade, wherein the blade is at least partially retracted into a recess when the blade is in the receiving position, and wherein the recess is located on an inner surface of the tissue treatment area of the debridement conduit.

8. The system of claim 1, further comprising a handle coupled to the cutting head, the handle comprising a cutting switch movable between a first position and a second position, wherein the cutting member moves from the receiving position to the cutting position when the cutting switch is moved from the first position to the second position.

9. The system of claim 1, further comprising a handle coupled to the cutting head, the handle comprising a reduced-pressure switch, wherein an amount of reduced pressure transferred to the cutting head changes as the reduced-pressure switch moves.

10. The system of claim 1, further comprising:
a fluid source fluidly coupled to the cutting head for supplying fluid to the cutting head; and
a handle coupled to the cutting head, the handle comprising a fluid switch, wherein the fluid is transferred from the fluid source to the cutting head in response to the fluid switch.

11. The system of claim 1, further comprising a fluid source for supplying fluid to the cutting head, wherein the cutting head further comprises a fluid supply conduit, the fluid supply conduit configured to transfer fluid to the tissue site during treatment of the portion of tissue.

12. (canceled)

13. (canceled)

14. A method for cutting a portion of tissue from a tissue site of a patient using reduced pressure, the method comprising:
providing a debridement conduit having a distal end and a proximal end;
causing the portion of tissue to enter the distal end of the debridement conduit in response to applying the reduced pressure to the portion of tissue via the debridement conduit; and
cutting the portion of tissue in the debridement conduit from the tissue site at a location that is displaced from the distal end of the debridement conduit.

15. The method of claim 14, wherein cutting the portion of tissue in the debridement conduit comprises:
activating a trigger operatively connected to a cutting member adjacent the distal end of the debridement conduit; and
moving the cutting member from a receiving position to a cutting position in response to activating the trigger.

16. The method of claim 14, further comprising adjusting the extent to which the portion of tissue enters the distal end of the debridement conduit by adjusting an amount of the reduced pressure applied to the portion of tissue via the debridement conduit.

17. The method of claim 14, further comprising:
increasing an amount of the reduced pressure being applied to the portion of tissue via the debridement conduit to increase the portion of tissue entering the distal end of the debridement conduit.

18. The method of claim 14, further comprising:
decreasing an amount of the reduced pressure being applied to the portion of tissue via the debridement conduit to decrease the portion of tissue entering the distal end of the debridement conduit.

19. The method of claim 14, wherein the debridement conduit forms part of a cutting head and wherein a portion of the debridement conduit adjacent to the distal end comprises a tissue treatment area, the cutting head comprising:
an opening to the tissue treatment area at the distal end of the debridement conduit, wherein the tissue treatment area of the debridement conduit is adapted to receive the portion of tissue through the opening under reduced pressure; and
a cutting member associated with the debridement conduit at the tissue treatment area, the cutting member movable from a receiving position to a cutting position to cut the portion of tissue from the tissue site while the portion of tissue resides in the tissue treatment area of the debridement conduit.

20. (canceled)

21. The method of claim 14, further comprising:
using reduced pressure to remove the portion of tissue after the portion of tissue has been cut; and
supplying a liquid to the distal end of the debridement conduit.

22. An apparatus for cutting a portion of tissue from a tissue site of a patient using reduced pressure, the apparatus comprising:
a cutting head for placement at the tissue site, the cutting head comprising:
a debridement conduit having a distal end and a proximal end, wherein a portion of the debridement conduit adjacent to the distal end comprises a tissue treatment area,
an opening to the tissue treatment area formed at the distal end of the debridement conduit, wherein the tissue treatment area of the debridement conduit is adapted to receive the portion of tissue through the opening when under reduced pressure, and
a cutting member associated with the debridement conduit at the tissue treatment area, the cutting member movable from a receiving position to a cutting position to cut the portion of tissue from the tissue site while the portion of tissue resides in the tissue treatment area of the debridement conduit.

23. The apparatus of claim 22, wherein the tissue treatment area of the debridement conduit comprises an enlarged disc having a width (W₁), wherein the debridement conduit comprises an extender portion having a width (W₂) that is adjacent the proximal end of the cutting head, and wherein W₁ is greater than W₂.

24. The apparatus of claim 22, wherein the tissue treatment area of the debridement conduit comprises an enlarged cushion portion having a width (W₁), wherein the debridement conduit comprises an extender portion having a width (W₂) that is adjacent to the proximal end of the cutting head, and wherein W₁ is greater than W₂.

25. The apparatus of claim 22, wherein:
the tissue treatment area of the debridement conduit comprises an enlarged disc having a width (W₁);
the debridement conduit comprises an extender portion, having a width (W₂), adjacent the proximal end of the cutting head;
W₁ is greater than W₂; and
the cutting head further comprises an elastic cover adapted to cover at least a portion of the enlarged disc, the elastic cover having an aperture at a first end for exposing the opening when the elastic cover is placed on the enlarged disc.

26. (canceled)

27. The apparatus of claim 22, wherein the cutting member is a blade, wherein the blade is at least partially retracted into a recess when the blade is in the receiving position, and wherein the recess is located on an inner surface of the tissue treatment area of the debridement conduit.

28. The apparatus of claim 22, further comprising a handle coupled to the cutting head, the handle comprising a cutting switch movable between a first position and a second position, wherein the cutting member moves from the receiving position to the cutting position when the cutting switch is moved from the first position to the second position.

29. The apparatus of claim 22, further comprising:
a handle coupled to the cutting head, the handle comprising a reduced-pressure switch movable between a first position and a second position, wherein an amount of reduced pressure transferred to the cutting head changes as the reduced-pressure switch moves between the first and second positions.

30. The apparatus of claim 22, wherein the cutting head further comprises a fluid supply conduit, the fluid supply conduit operable to transfer fluid from a fluid source to the tissue site during treatment of the portion of tissue.

31.-39. (canceled)
